(19) United States
(12) Patent Application Publication
(10) Pub. No.: US 2010/0275912 A1
(43) Pub. Date: Nov. 4, 2010

(54) AEROSOL DEVICE

(75) Inventors: Amar Lulla, Maharashtra (IN); Xersees Rao, Maharashtra (IN)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: CIPLA LIMITED

(21) Appl. No.: 12/312,606

(22) PCT Filed: Nov. 20, 2007
(86) PCT No.: PCT/GB2007/004432

§ 371 Date: Dec. 8, 2009

(30) Foreign Application Priority Data
Nov. 20, 2006 (IN) 1914/MUM/2006
Aug. 24, 2007 (IN) 1635/MUM/2007

(51) Int. Cl. A61M 15/00

(52) U.S. Cl. 128/203.12

(57) ABSTRACT

The invention relates to an aerosol device. More specifically the invention relates to an aerosol device for a Metered Dose Inhaler. The aerosol device (76,176) comprises a container (66,166) with an opening at one end, a self sealing valve assembly (60,160) supported within the container (66,166), and a cap member (72,172) for maintaining the valve assembly (60,160) in the container (66,166), wherein the cap member (72,172) is secured to the container (66,166) in order to retain the valve assembly (60,160) in position.

The present invention relates to an aerosol device and more specifically, but not exclusively, to an aerosol device for a Metered Dose Inhaler, and to a valve assembly for said aerosol device.

The term aerosol is considered to encompass all types of pressurized containers used for delivery of aerosolized products meant for a variety of medical and non-medical applications including, but not limited to, drugs, cosmetics (deodorants, hair sprays, hair mousses, shaving creams), perfumes, air fresheners, insect repellents, cleaning agents, paints, lubricants and the like. Aerosol devices may deliver aerosolized ingredients in an uneven or continuous manner delivering varying quantities per actuation, or in a uniform manner delivering predetermined identical quantities, or doses, per actuation.

A Metered Dose Inhaler (MDI) is a dispenser designed to deliver a specific dose of medication to a user with each usage.

Typically, aerosol devices comprise a container, and a valve crimped on the container. The valve is fitted to the body of the container by crimping a valve ferrule against the body of the container with an intermediate seal made of an elastomeric material compressed between the body and the ferrule.

Crimping is a method of hermetically sealing the body of a container and a valve by applying pressure to the valve assembly against the container. However, the crimping process can lead, to inefficient and improper sealing of the body of the container and the valve. Conventional pressurized aerosol devices are prone to leakages resulting from improper sealing of the valve and the body of the container.

The problem of leaks is especially significant in the case of medicinal aerosol formulations. In medicinal aerosol formulations it is important to ensure that an appropriate concentration of the drug in the container is maintained throughout the entire usage period. Leakage of the propellants in such devices can lead to non-uniform and unpredictable drug delivery, which is a serious problem with these devices.

It is common in many aerosol devices to use thermoplastic elastomers as an additional means to obtain a more effective seal. However, in aerosols meant for medicinal purposes this technique has further drawbacks. Contact of the medication with an elastomer greatly increases the chances of drug contamination through the process of leaching. If the elastomeric elements are not incorporated, in an effort, to avoid this problem, the sealing of the aerosol device through crimping is compromised, leading to increased leakage problems as discussed above.

Aside from the difficulty in obtaining an efficient seal, the process of positioning a valve in place and crimping around it to form a seal and close the container is time consuming. The device has to be assembled at the site of the filling station, which adds a step in the overall production line thereby increasing the overall cost and time required for the production. Furthermore, frequent testing of the devices is conducted throughout the manufacturing process to ensure that leaks are not present. This all requires extra skilled man-power and machinery, thereby increasing the overall cost and time required for the production.

Numerous attempts have been made to make leakage resistant aerosol devices. Various known aerosol devices incorporate an intermediate seal, either in the form of a ferrule gasket placed and compressed between the top edge of the body of the container and the opposing surface of the ferrule gasket, or of an O-ring placed around the exterior of the body of the container and compressed between the body of the container and an annular flange of the valve ferrule. Other approaches include incorporating gaskets made up of material of varying durometer values, in an attempt to achieve more effective sealing. However, the problems discussed above still remain.

There is therefore a need for an aerosol device that is leakage-resistant, economical to produce and minimizes the use of elastomeric materials during construction.

It would also be beneficial if a valve assembly could be provided for an aerosol device which enabled the aerosol device to be more easily sealed, while also allowing more efficient production of the aerosol device.

It is also desirable, especially in the case of medical aerosols, to have some indication of the amount of a substance which remains for use in an inhaler at any time. It is very important, for example, that asthma sufferers can be sure that they have a sufficient number of doses remaining in an inhaler, especially if they are likely to be unable to readily obtain refills. In Metered Dose Inhalers it is common to include a dedicated dose counter, actuated by the movement of a canister between a stowed and a discharge position, to signify the number of doses expelled or remaining. These dose counters come in a variety of forms, both mechanical and electrical, and are commonly formed as part of the housing of an inhaler device or are mounted in some way to a canister. While the counting of doses is generally reliable, no counter is infallible. Doses may be counted when no medicament is dispensed or, more significantly, missed when a dose is dispensed. Furthermore, there is the possibility that a counter may be inadvertently re-set before a container is exhausted, leaving a user with no indication of the remaining number of doses contained therein. The dose counters also inevitably increase the size, complexity and cost of the inhaler devices on which they are provided.

Conventional aerosol devices have further disadvantages. In many cases, due to the shape of the container used, it is not possible for the container to dispense every last drop of a substance to be delivered. This is wasteful in all applications, but is most significant where a medicament is to be delivered by the aerosol device. Canisters for use with Metered Dose Inhalers are designed to deliver a certain number of controlled doses of medicament to a user. The devices are commonly employed in inhaler apparatus to treat asthma and similar complaints. Since, in existing canisters, there is often a quantity of the medicament which can not be delivered, the container is routinely over-filled (in some cases by up to fifteen percent). This not only increases the amount of medicament, propellant and the size of container required for a given number of doses, but also complicates the calculation of the amount of medicament required. If one could be sure of complete exhaustion of a container during use, then less medicament could be used with a smaller container, the filling of which would be simplified because only the number of doses and size of each dose would need to be known in order to determine the size/volume of container required.

It is an object of the present invention to provide an aerosol device, the manufacture of which does not involve the step of crimping. It is a related object of the present invention to provide an aerosol device which forms an efficient and substantially leak-proof seal with minimal use of elastomeric materials in areas where they may come in contact with a medicament contained in the device. It is a further object of this invention to provide an aerosol device capable of more efficient production.

It is a further object of the present invention to provide an aerosol device which allows a user to obtain a clear indication of the amount of substance that remains to be exhausted. It is also an object of the present invention to provide an aerosol device which allows for nearly complete exhaustion of the substance contained therein.

It is another object of the present invention to provide a valve for an aerosol device which also serves as a closure for the aerosol device. It is a related object of the present invention to provide a valve assembly for an aerosol device which allows for nearly complete exhaustion of the substance contained in the aerosol device.

All of the above objects are addressed by the aerosol device hereinafter described.

According to a first aspect of the present invention there is provided an aerosol device comprising a container with an opening at one end, and a valve assembly supported within the container. Preferably, sealing means are provided to create a seal between the valve and the container, and separate means are provided for retaining the valve assembly in place in the container. The means for maintaining the valve assembly in position may comprise a cap member.

In prior art devices the valve assembly itself is secured to the container by crimping to form a seal. By instead employing separate sealing and securing means the crimping required, especially in the area of the valve, is minimised. Preferably, the cap member is secured to the container in order to retain the valve assembly in position.

The dimensions of the opening in said one end of the container are preferably substantially equal to the internal dimensions of the container adjacent the opening in the end of the container. This means that one end of the container will be entirely open.

In one embodiment, the valve assembly is located in an open end of a, preferably substantially cylindrical, container. Peripheral sealing is preferably provided between the outside of a valve assembly and the inside of a container. The cap member holding the valve assembly in place may be positioned around the outside of the container, or may be attached to the inside of the container, and may be crimped or otherwise attached to the container. Due to the peripheral sealing of the valve assembly, there is no need for the means of attaching the cap member to also serve as a seal. Peripheral sealing of this type is preferable to so called ‘face sealing’, where the valve member is sealed only against a planar face of a canister. A better seal can be achieved from the periphery of a valve assembly than from simple interaction of two substantially planar surfaces. Face sealing typically also requires crimping of a ferrule with a gasket and a valve, which can cause damage to the neck of the can. The peripheral sealing method obviates the need for crimping of this type. This also results in an aerosol device which is more simply and efficiently manufactured.

Peripheral sealing is most easily achieved in an assembly where the container and valve assembly are both cylindrical in shape. Sealing elements such as elastomeric rings may be employed to achieve the seal between an exterior part of the valve element and an interior part of the container. If, as is preferable, the valve assembly closes the entire open end of the container, the elastomeric components are positioned so as not to come into direct contact with the contents of the container. The valve assembly may rest on a ledge portion provided on the interior surface of the container, and be prevented from lifting off the ledge portion by the cap member. In this instance, an aperture should be provided in the cap member to allow a valve stem of the valve arrangement to pass therethrough.

Alternatively, the cap member may close the opening in the end of the container. Again, the container is preferably substantially cylindrical, especially adjacent the opening, and the cap member may be secured to the outside of the container, or the inside. In this case a further feature should be included on the cap member to support the valve assembly in position within the container. This could take the form of a stalk, extending from the cap member and through the container when the container and cap member are assembled.

A valve stem of the valve assembly could, in this configuration, extend through a further aperture provided in the end of the container opposite the opening.

All of the above aspects may preferably be applied to aerosol devices containing medicament to be delivered to a patient. The valve assembly is preferably pre-assembled before it is inserted into the container. The cap member may be crimped to the container, snap-fit or joined by any other suitable means. Over-molding may also be employed at the join between the cap and the container to ensure that the cap member remains in place.

The container may be formed, at least in part, from a transparent or translucent material such as glass or a plastics material, for example a polysulphone. The container is also preferably substantially cylindrical, and alternatively, or additionally, may comprise a flat end portion on the interior to prevent pooling of a substance in the container. The flat end portion may be provided by the valve assembly.

According to a second aspect of the present invention there is provided an aerosol device comprising a container formed at least in part from transparent or translucent material. An aerosol device is thereby provided that allows a user to see how much substance remains therein. The need for other indicating means such as dose counters is thereby avoided.

The container is preferably transparent, but may be merely translucent. For the purposes of the application, ‘transparent’ is taken to mean that the container is capable of transmitting visible light so that it can be seen through without any noticeable diffusion. ‘Translucent’ is taken to mean imperfectly transparent; visible light is transmitted with a degree of diffusion making it more difficult to perceive distinct images.

The container preferably comprises glass or a plastics material. Polysulphone (or polysulfone) plastics such as polysulphone (PSU) and polyethersulphone (PES) give a high degree of rigidity, are very stable chemically and mechanically and have excellent thermal, electrical and creep resistant properties over a wide temperature range. The polysulphone plastics, which comprise a number of repeating sulphone monomer units, are also capable of providing a high degree of clarity, and are suitable for use under high pressures, making them particularly suitable for the manufacture of the container.

Preferably, the number of monomer units making up the polysulphone plastic is in the range from around 8000 to around 26000. More specifically, the number of monomer units may range from around 10000 to around 22000, for example 22000. Alternatively, the number of monomer units may range from around 12000 to around 18000, more preferably from around 14000 to around 16000, for example 16000.

The sulphone monomer may comprise the following moiety

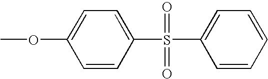

In particular, a polysulphone having the chemical formula

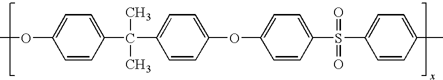

is preferred. The polysulphone shown is an amorphous and clear high-performance plastic with a light-brownish transparency. The polysulphone may have a molecular weight of around 35000. Alternatively, the molecular weight may range from around 17500 to around 57000, more specifically from around 22000 to around 48000, more specifically from around 26250 to around 39500, still more specifically from around 30500 to around 35000.

The container may further be provided with markings, such as etchings, to indicate the amount of a substance, either in terms of volume or of a number of doses of a formulation, expelled from or remaining in the container.

The container may also comprise an internal volume having a substantially cylindrical shape and/or flat end portions. The simple internal shape of the container and its flat end portions minimise the chances of a substance forming pools (ullage) or otherwise being prevented from being expelled from the container. One or more of the flat surfaces may be formed by a part of a valve assembly. To maximise the chances of expelling every drop of a substance, channels or suitable apertures should be provided on or immediately adjacent the flat end portion to provide access to a valve assembly.

If the container is substantially cylindrical in shape, providing a constant internal cross-sectional area, the level of substance visible through the wall of the container will be directly proportional to the volume of substance remaining in the container. This provides a clear and simple indication of the amount of substance remaining to be expelled. The container may be entirely transparent or translucent, or parts of the container may be opaque, so as to not let visible light to pass through.

All of the above aspects may preferably be applied to aerosol devices containing medicament to be delivered to a patient, specifically for use in a Metered Dose Inhaler (MDI).

A further aspect of the present invention provides a valve assembly for an aerosol device, the valve assembly being receivable in an opening provided in a container of the aerosol device to close said opening, wherein means are provided on the valve assembly to form a seal between the valve assembly and a container.

The valve assembly is preferably substantially cylindrical, so as to be locatable in an open end of a substantially cylindrical container. Peripheral sealing means may then be provided around the entire periphery of the valve assembly, to provide a seal between the outside of the valve assembly and the inside of the container. Peripheral sealing of this type is preferable to so called ‘face sealing’, where the valve member is sealed only against a planar face of a canister. A better seal can be achieved from the periphery of a valve assembly than from simple interaction of two substantially planar surfaces. Face sealing typically also requires crimping of a ferrule with a gasket and a valve, which can cause damage to the neck of the can. The valve assembly can be held in place by a cap, positioned around and crimped or otherwise attached to the outside of the container. Due to the valve assembly closing the entire opening of the container, and due to the peripheral sealing, there is no need for the means of attaching the cap member to also serve as a seal. This results in an aerosol device which is more simply and efficiently manufactured.

Peripheral sealing is most easily achieved in an assembly where the container and valve assembly are both cylindrical in shape. Sealing elements such as elastomeric rings may be employed to achieve the seal between an exterior part of the valve element and an interior part of the container.

The valve assembly preferably has a flat portion, perhaps formed by a body part of the valve assembly, which, in use, faces towards the interior of a container. The flat end portion minimises the chances of a substance forming pools or otherwise being prevented from being expelled from the container. To maximise the chances of expelling every drop of a substance, channels or suitable apertures should be provided on or immediately adjacent the flat surface to provide access to the interior of the valve assembly.

The component parts of the valve assembly may be arranged in a valve body and contained therein by a sealing piece, which may be snap-fit together, or joined together by other suitable means. This provides a self contained valve assembly ready for insertion into a suitable container. The component parts of the valve assembly may comprise, among others, a valve stem, a spring and a valve chamber. A space is preferably provided between the valve stem and valve chamber, to contain a volume of substance comprising one ‘dose’ for delivery when the valve is activated.

The component parts of the valve assembly may further comprise a seat gasket positioned between the valve chamber and the sealing piece. The seat gasket preferably surrounds the valve stem and is capable of sealing an aperture provided in the valve stem. The valve stem is preferably movable, for example slidable, within the valve chamber. By restricting the movement of the seat gasket, the aperture in the valve stem can be sealed and unsealed as the valve stem moves within the valve chamber.

The valve assembly is particularly suitable for application to aerosol devices containing medicament to be delivered to a patient, for example in the form of MDI canisters.

Aspects of the invention will be better understood with reference to the following detailed description of two preferred embodiments. The detailed description is included by way of example only, and is not intended to limit the protection sought. Throughout the detailed description reference is made to the accompanying drawings, in which:

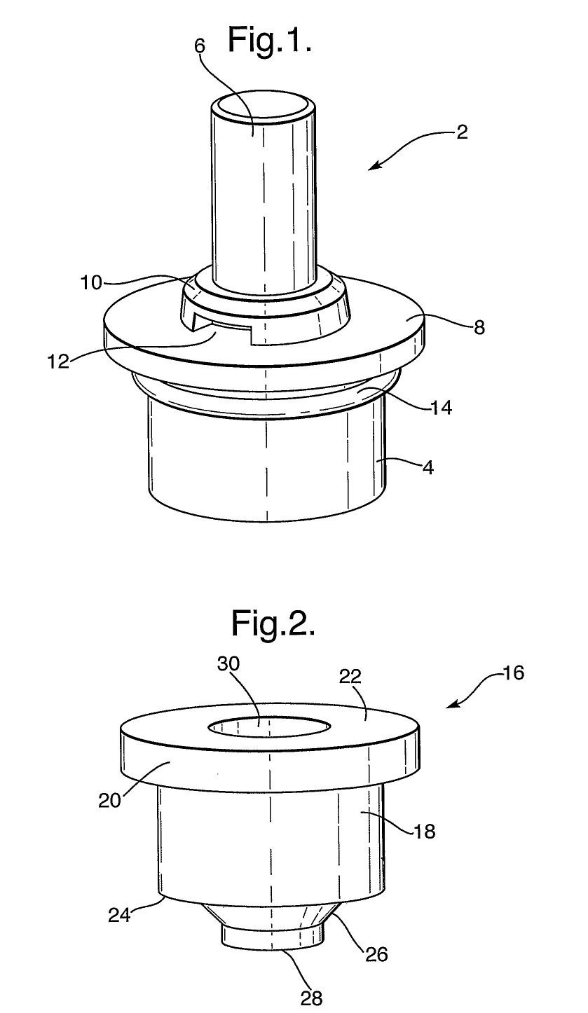

FIG. 1 is a perspective view of a valve body from a valve assembly of an aerosol device according to a first embodiment of the present invention;

FIG. 2 is a perspective view of a valve chamber from the valve assembly of said device according to a first embodiment of the present invention;

FIG. 3 is a perspective view of a valve stem from a valve assembly of said device according to a first embodiment of the present invention;

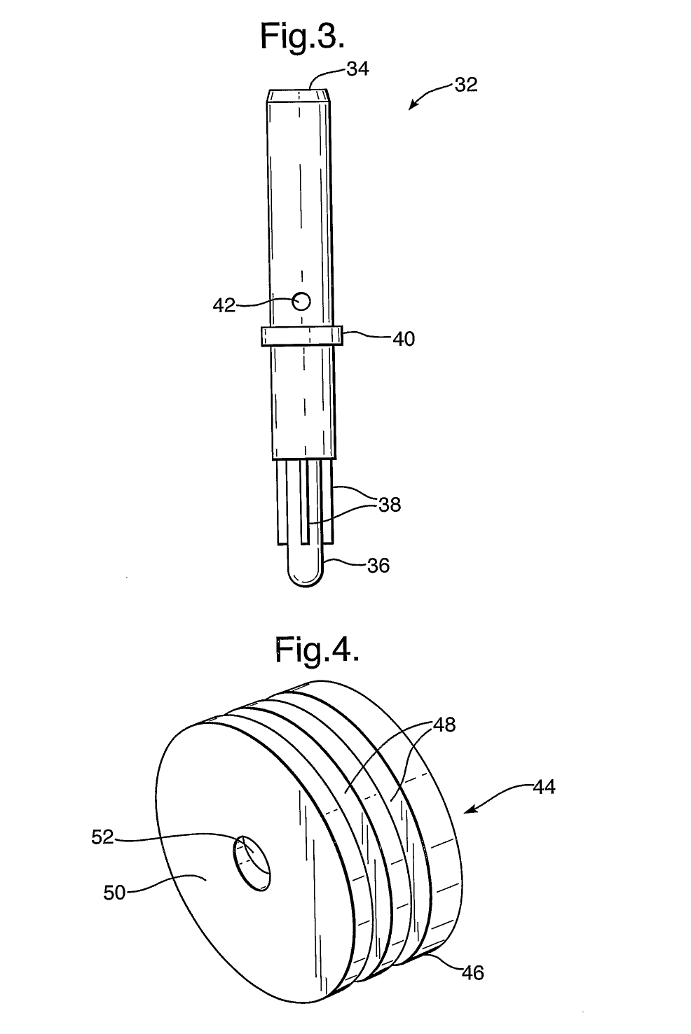

FIG. 4 is a perspective view of a sealing piece from a valve assembly of said device according to a first embodiment of the present invention;

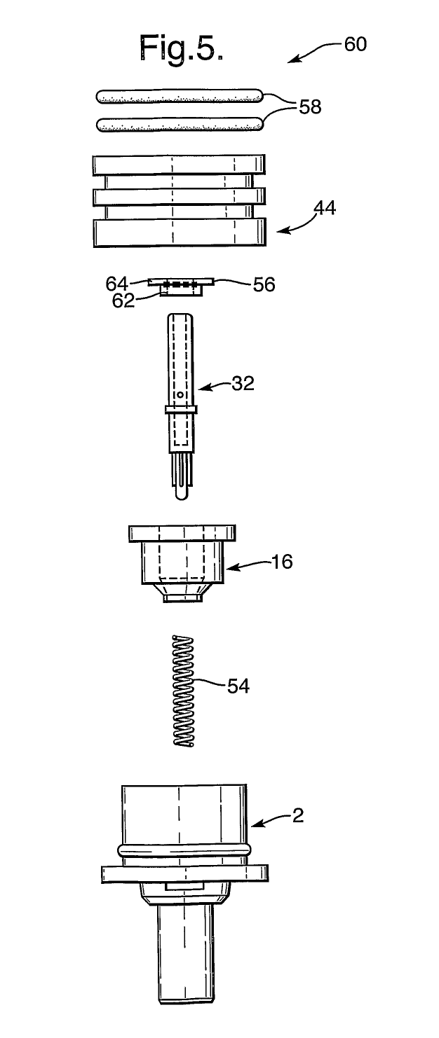

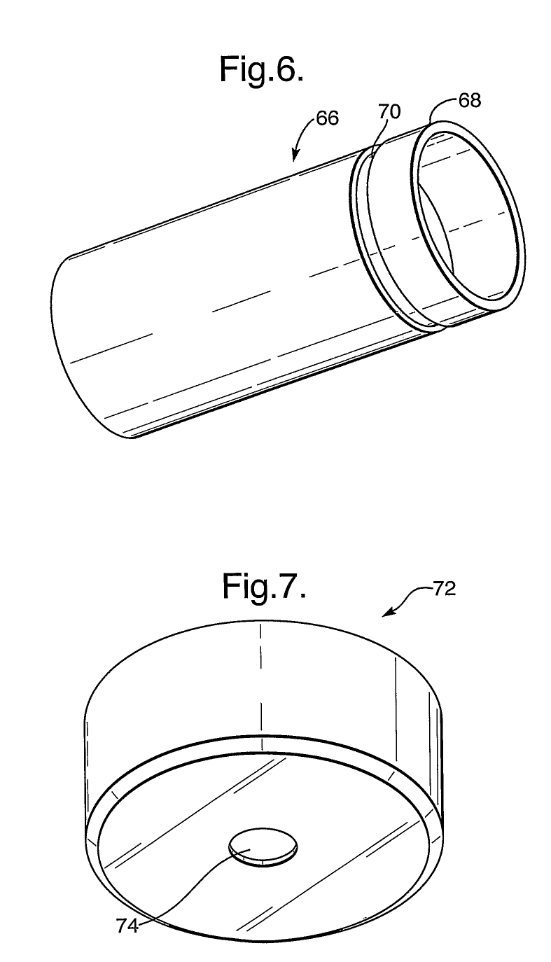

FIG. 5 is an exploded view of a valve assembly of said device according to a first embodiment of the present invention, including the parts shown in FIGS. 1-4;

FIG. 6 is a perspective view of a container of said device according to a first embodiment of the present invention;

FIG. 7 is a perspective view of an end cap of said device according to a first embodiment of the present invention;

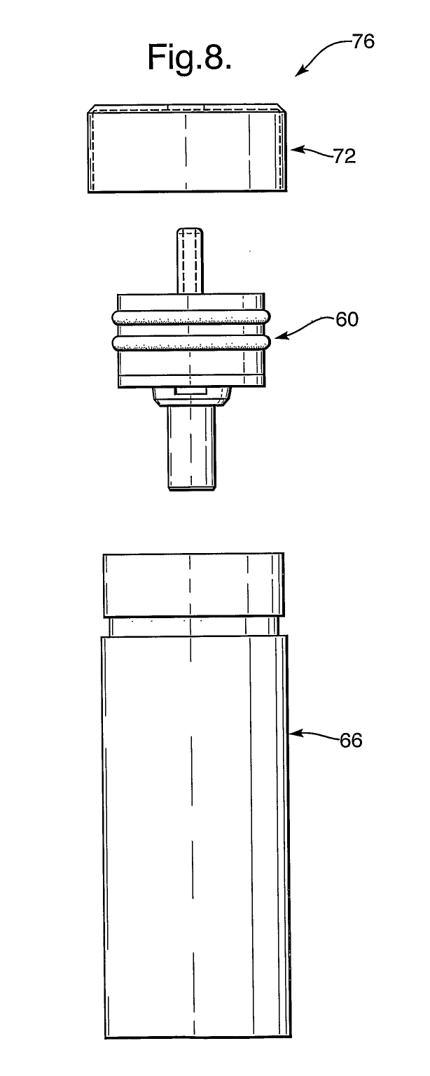

FIG. 8 is an exploded view of said aerosol device according to a first embodiment of the present invention;

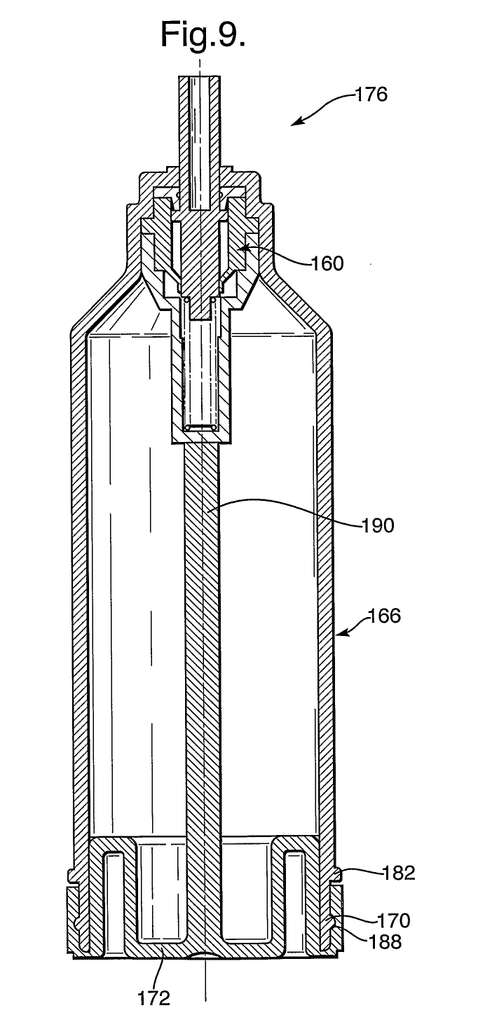

FIG. 9 is a cross-sectional view of an aerosol device according to a second embodiment of the present invention;

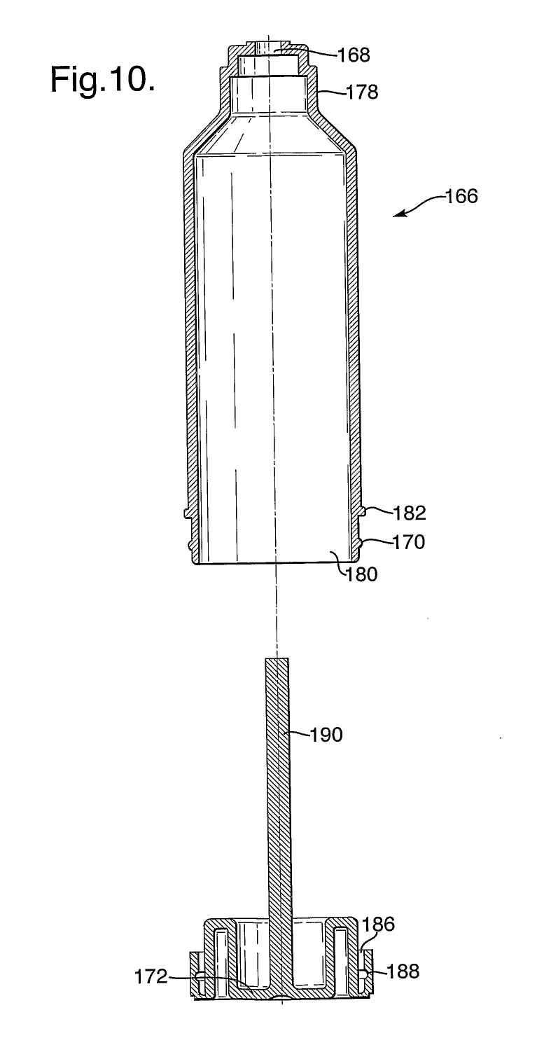

FIG. 10 is a cross-sectional view of a tubular container and base cap of the aerosol device of FIG. 9;

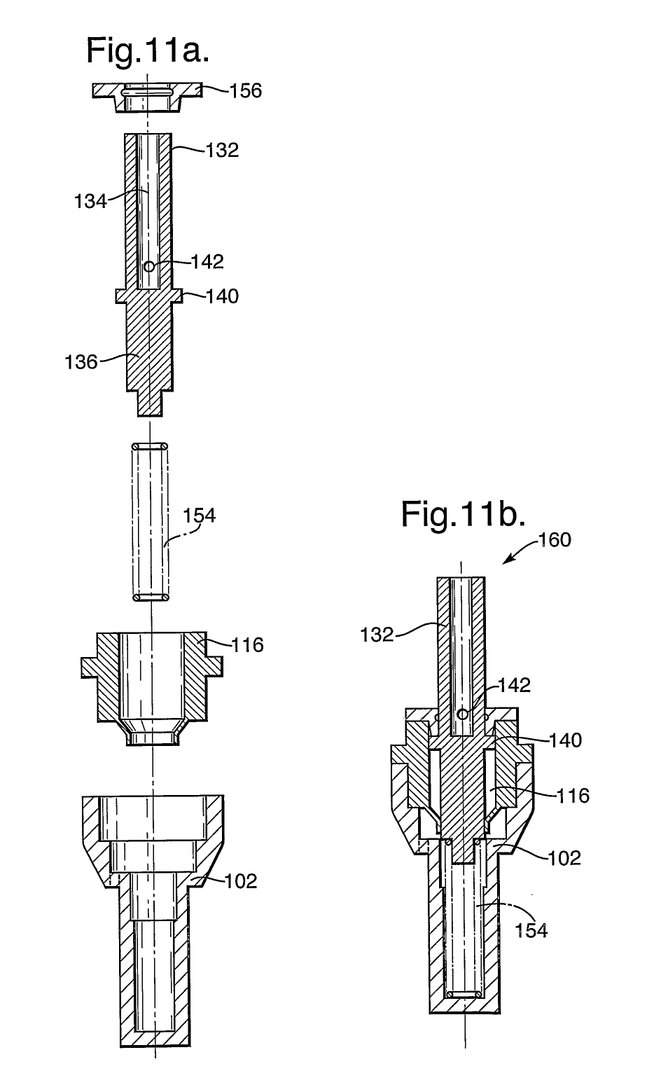

FIG. 11a is a cross-sectional exploded view of a valve assembly from the aerosol device of FIG. 9;

FIG. 11b is a cross-sectional view of a valve assembly from the aerosol device of FIG. 9 in its assembled state;

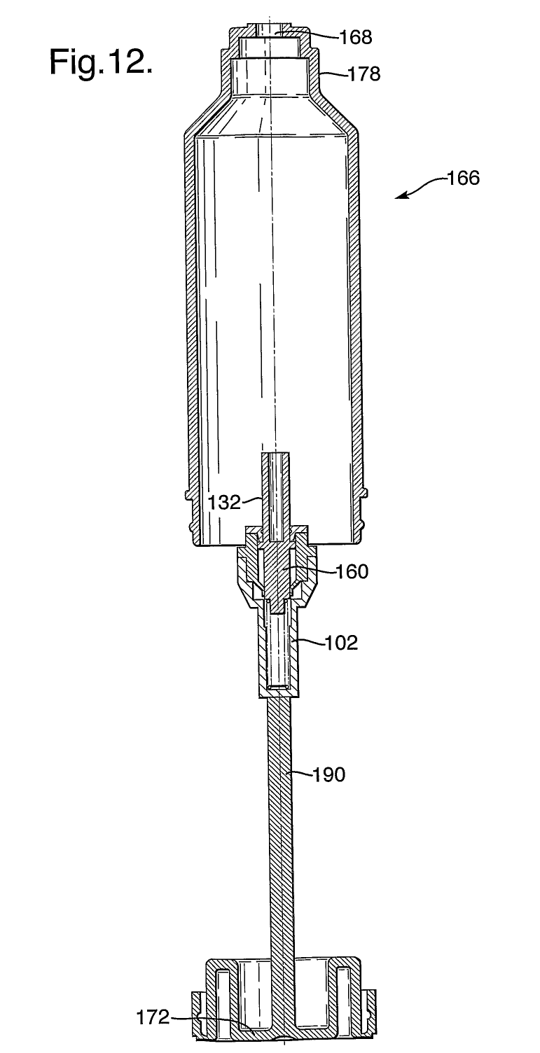

FIG. 12 is a cross-sectional view of a tubular container and base cap of the aerosol device of FIG. 9, with the valve assembly of FIG. 11b mounted on a stalk extending from the base cap; and

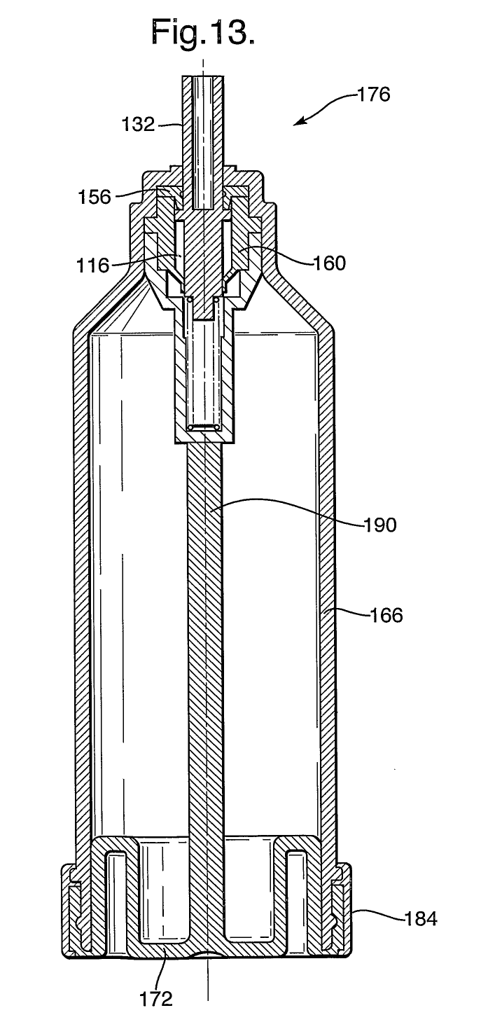

FIG. 13 is a cross-sectional view of an aerosol device according to a second embodiment of the present invention, with over-molding.

A first embodiment of the present invention is shown in FIGS. 1 to 8 of the accompanying drawings.

The valve body 2 of FIG. 1 comprises an open hollow tubular section 4 at one end, and an enclosed tubular section 6, of a smaller diameter, at the other. On the interior of the valve body 2, the tubular sections 4,6 join up such that the valve body 2 is substantially hollow. Between the two tubular sections 4,6, to the exterior of the valve body 2, a circular disc shaped flange 8 is provided. On the side of the flange 8 adjacent the closed tubular section, a raised area 10 is provided, with two apertures 12 on opposite sides thereof. The apertures 12 are in the form of slots which run against the flat surface of the flange 8 and provide fluid communication between the outside and the inside of the valve body 2. A circumferential ridge 14 is provided to the exterior of the open tubular section 4 of the valve body 2.

FIG. 2 shows a valve chamber 16 which, in use, fits inside the open tubular section 4 of the valve body 2 of FIG. 1. The valve chamber 16 has a main cylindrical section 18 with an external diameter substantially equal to the internal diameter of the open tubular section 4 of the valve body 2. At one end of the cylindrical section 18 there is a circular flange 20 which is sized to rest on a lip provided on the interior of the open tubular section 4 of the valve body 2, adjacent the open end. Accordingly, the flat end section 22 of the flange 20 finishes flush with the open end of the open tubular section 4 of the valve body 2 when the valve body 2 and valve chamber 16 are assembled.

At the end of the cylindrical section 18 opposite the flange 20, a square shoulder 24 is provided. Radially inward from the square shoulder, a truncated cone section 26 narrows the outer diameter of one end of the valve chamber 16 to a circular opening 28 which leads to an axial tubular bore 30 running through the centre of the valve chamber 16, emerging at the centre of the flange 20. The opening 28 at the end of the valve chamber 16 is of a smaller diameter than the bore 30 which runs through the valve chamber 16, and out of the centre of the flange 20.

FIG. 3 shows a preferred valve stem 32 which, in use, is positioned in the bore 30 of the valve chamber 16. The valve stem 32 is essentially a thin tubular part, with one end enclosed. The open end 34 of the tube is the part of the valve assembly through which a substance is expelled during use. The closed end 36 of the tube is opposite to the open end 34, and is of a smaller diameter. Four radially extending fins 38 are provided on a part of the closed end 36 of the valve stem 32, spaced slightly from the end. The fins 38 give an effective external diameter larger than the closed end 36 of the valve stem 32, but slightly smaller than its main hollow section. A flange 40 is provided approximately half way along the valve stem 32. To the side of the flange 40 adjacent the open end 34 of the valve stem 32 there is a small aperture 42. The aperture 42 provides fluid communication with the hollow section of the valve stem 32.

The external diameter of the hollow part of the valve stem 32 is slightly smaller than that of the opening 28 at the end of the valve chamber 16 adjacent the truncated cone section 26. However, the diameter of the flange 40 is slightly larger than the opening 28 at the end of valve chamber 16, but slightly smaller than the internal diameter of the bore 30 which runs through the valve chamber 16. The significance of this will be explained below.

The sealing piece 44 shown in FIG. 4 comprises a section of tube 46 which is largely closed at one end. In use, it fits over the open hollow tubular section 4 of the valve body 2. The internal diameter of the sealing piece 44 is substantially the same as the external diameter of the open hollow tubular section 4 of the valve body 2, to ensure a slight interference fit therebetween. The interior surface (not shown) of the sealing piece 44 further comprises a circumferential channel to engage with the circumferential ridge 14 of the valve body 2. Two further circumferential channels 48 are provided on the outside wall 46 of the sealing piece 44 to accommodate elastomeric sealing rings (not shown). The largely closed end of the sealing piece 44 is provided as a flat disc-like end 50 having a small circular hole 52 in the centre. The small hole 52 is just large enough for the hollow part of the valve stem 32 to pass through.

The interaction of the various parts of the valve assembly described in FIGS. 1-4 will now be described in relation to the exploded view of FIG. 5.

FIG. 5 shows a front view of the various components that make up a valve assembly 60 in accordance with the present invention. Along with the valve body 2, valve chamber 16, valve stem 32 and sealing piece 44 described previously, the assembly further comprises a spring 54, a seat gasket 56 and two elastomeric sealing rings 58. For the sake of simplicity in the drawings, the reference numbers for various individual parts of the components shown in FIG. 5 are not shown on the exploded view. The reader is referred back to FIGS. 1-4.

The assembly of the valve assembly 60 is relatively straightforward. The spring 54 is inserted into the valve body 2 where it locates in the enclosed tubular section 6. The valve stem 32 is inserted into the valve chamber 16 so that the closed end 36 of the valve stem 32 passes through the opening 28 in the valve chamber 16. The seat gasket 56 is positioned on the valve stem 32 so that it abuts the flange 40. In this position, the seat gasket 56 covers the aperture 42 in the side of the valve stem 32. The seat gasket has a stepped cross section, the smaller diameter part 62 of which sits against the flange 40 on the valve stem 32, and is sized so as to fit inside the bore 30 of the valve chamber. The larger diameter part 64 rests on top of the flange 20 of the valve chamber 16.

The valve chamber 16, with the valve stem 32 and seat gasket 56 in place, is then lowered into the valve body 2. The closed end 36 of the valve stem 32 engages with the spring 54, which is supported against the radially extending fins 38 of the valve stem 32. Once the valve chamber 16 has been pushed firmly into position inside the valve body 2, the sealing piece 44, with elastomeric sealing rings 58 in position in its external circumferential channels 48, is pushed into place around the outside of the valve body 2 such that the circumferential ridge 14 of the valve body 2 engages with the internal circumferential channel (not shown) of the sealing piece 44. The valve assembly 60 then forms a complete self contained unit.

Because the hollow section of the valve stem 32 is of a slightly greater diameter than the effective diameter of the radially extending fins 38, the valve stem 32 tends to naturally sit in the valve chamber 16 such that only the closed end 36 of the valve stem 32 and the radially extending fins 38 extend beyond the opening 28 in the valve chamber 16. The hollow portion of the valve stem 32 is capable of passing through the opening 28, but due to its greater diameter some further impetus (force) is require to make it do so. The result of this is that, when the valve assembly 60 is in its complete state, a force applied to the open end 34 of the valve stem must overcome not only the resistive force of the spring 54, but also the additional resistance caused due to the change in diameter of the valve stem 32. This provides a greater initial resistance than the spring 54 alone, which is helpful in avoiding accidental actuation of the valve assembly 60. When the external force is removed, the restoring force of the spring 54 is sufficient to return the valve stem 32 to its rest position. The flange portion 40 of the valve stem 32 is too large to pass through the opening 28 in the valve chamber 16, and so provides a stop to avoid over actuation of the valve stem 32.

The container 66 shown in FIG. 6 is configured to receive the valve assembly 60 described above. The container 66 has a simple cylindrical shape and is enclosed at one end only. The open end 68 of the container 66 is sized to receive the valve assembly 60. The internal diameter of the open end 68 is substantially the same as, or slightly smaller than, the external diameter of the complete valve assembly 60, including the elastomeric sealing rings 58. A lip (not shown) is provided on the interior of the container 66 to support the valve assembly 60. The lip is positioned such that the flat disc like end 50 of the sealing piece 44 of the valve assembly 60 is positioned flush with the opening of the container 66 once assembled. A circumferential groove 70 is provided in the outside wall of the container 66 adjacent the open end 68. The container 66 is formed of an at least partially transparent plastics material, in this case a polysulphone, so that a user can readily see how much substance is in the container at any given time.

The metal end cap 72 shown in FIG. 7 is designed to be placed over the open end 68 of the container 66 once the valve assembly 60 is in place, in order to form a complete aerosol device. The end cap 72 takes the form of a cylinder with one flat substantially closed end face with a hole 74 provided in the centre. The end cap 72 can be crimped or attached by some other means to the container 66. Where crimping is used, the forces involved can be much less than in typical aerosol manufacture. This is because the end cap 72 of the present invention need not serve any sealing purpose, but merely has to retain the valve assembly 60 in position in the container 66. Furthermore, there is no need to apply any crimping force directly to the valve assembly 60, so the possibility of causing damage to the valve is minimised.

The final assembly of the aerosol device 76 will be understood with reference to FIG. 8. FIG. 8 shows front views of the container 66, the end cap 72 and the complete valve assembly 60.

The valve assembly 60 is inserted into the open end 68 of the container 66, such that it rests on the internal lip (not shown) provided therein. The elastomeric sealing rings 58 of the valve assembly 60 form a seal around the entire circumference of the valve assembly 60. The end cap 72 is then placed over the end of the container 66 with the valve stem 32 extending out through the hole 74, and crimped into the circumferential groove 70 provided on the container 66 to retain the valve assembly 60 in place. If better joining is required then further elastomeric elements may be incorporated between the end cap 72 and the container 66. Alternatively, or additionally, one or more further circumferential grooves may be provided in the exterior surface of the container.

Once the aerosol device 76 is complete, it can be filled through the valve ready for use. It should be noted that, although the assembly of parts of the aerosol device 76 is shown in the drawings with the valve positioned at the top, in use it is likely that the aerosol device will be inverted from this position. In this regard, it is significant that one side of the flange disc 8 will provide a flat base for the inside of the aerosol device. Referring back to FIG. 1, the flat flange disc has, on one side, a raised portion 10 with two slot shaped apertures 12 therein. The slots 12 run flat along the face of the flange 8 into the interior of the valve body 2. Given that the face of the flange 8 will form a flat base of the aerosol device 76 when in use, the positioning of the slots 12 allows for all of the contents of the aerosol device 76 to be exhausted. There is nowhere in the device for a substance to ‘pool’ and not be in communication with an aperture.

Once the substance passes through the slots 12 in the valve body 2, it then moves into contact with the valve chamber 16. Because of the seating of the valve stem 32 in the opening 28 of the valve chamber 16, the substance is able to pass through the gaps between the radially extending fins 38 of the valve stem 32 and through the opening 28 in the valve chamber 16. A small amount of the substance is, therefore, present in the clearance volume between the exterior of the valve stem 32 and the interior of the valve chamber 16. This constitutes a single ‘dose’ to be exhausted from the aerosol device 76. The substance is prevented from leaving the clearance volume by the seat gasket 56, which seals not only the end of the bore 30 of the valve chamber 16, but also the small aperture 42 in the side of the valve stem 32.

When the valve stem 32 is subjected to an external force, it is pushed into the remainder of the valve assembly 60 against the force of spring 54. As the hollow part of the valve stem 32 passes through the opening 28 in the valve chamber 16, the valve chamber 16 is sealed off from the valve body 2 and, therefore, from the container 66. The movement of the valve stem 32 also causes the aperture 42 in the side of the valve stem to be uncovered, since the seat gasket 56 remains in place due to the interaction of its larger diameter part 64 with the flat surface 22 of the flange 20 of the valve chamber 16, as the valve stem 32 is depressed. This allows the substance contained in the clearance volume between the valve stem 32 and the valve chamber 16 to enter the hollow part of the valve stem through aperture 42, and be expelled through the open end 34 of the valve stem 32 in the form of a fine mist or spray.

FIGS. 9 to 13 show a second embodiment of the present invention.

FIG. 9 shows, in cross-section, an integrated aerosol device 176, in the form of an MDI canister, comprising a tubular container 166, a valve assembly 160, a base cap 172 and a stalk 190.

The tubular container 166 shown in FIG. 10 has a slender neck 178. The neck 178 is provided with an opening 168 at one end. Unlike more conventional aerosol devices, the distal end 180 of the tubular container 166, opposite the neck portion 178, is also open. A first annular ridge 170 is provided on the outer surface of the distal end 180 of the container 166. At least one further outward annular locking ridge 182 is provided on the outer surface of the container 166, spaced from the first annular ridge 170 in the direction of the neck 178 of the container.

FIG. 10 also shows a base cap 172 of the container aerosol device 176. The cross-sectional view shows that the base cap 172 is corrugated so as to form an annular channel 186 close to its periphery for receiving the wall of the tubular container 166. A groove 188 is provided within the channel 186 on the inner surface of the outer channel wall and is sized and positioned to receive the first annular ridge 170 provided on the container body 166. When the base cap 172 is inserted into the opening at the distal end 180 of the tubular container 166, the groove 188 and ridge 170 engage and the two parts snap-fit together as shown in FIG. 1 to form a leakage resistant sealing engagement. A stalk 190 provided on the base cap 172 extends into the container body 166, for reasons that will be explained later.

FIGS. 11a and 11b show a valve assembly 160 for an aerosol device 176 according to a second embodiment of the present invention. The individual parts of the valve are shown most clearly in the exploded view of FIG. 11a. The valve assembly 160 fits inside the neck 178 of the container 166 and comprises a valve chamber 116, a seat gasket 156, a valve stem 132, a valve body 102 and a spring element 154.

The valve stem 132 is provided with an orifice 142 in a side thereof, such that it can be displaced along the longitudinal axis inside the valve chamber 116. The valve stem 132 is also provided with an integrated annular stopper 140, which limits the vertical movement of the valve stem 132 within the valve chamber 116. As in the first embodiment of the invention, the valve stem 132 may be partially solid 136 and partially hollow 134. The valve stem 132 is typically tubular and the orifice 142 is typically formed in the hollow tubular region.

The seat gasket 156 has an aperture through which the valve stem 132 extends along the longitudinal axis outside the valve assembly 160 and also out of the tubular container 166 passing through the opening 168 in the neck 178 thereof.

The fully assembled valve assembly 160 is shown in cross-section in FIG. 11b. The assembly of the various parts and their interaction is essentially the same as for the valve assembly of the first embodiment. The valve body 102 contains the spring element 154, which is attached to valve stem 132. Whenever the spring 154 is compressed (during actuation of the aerosol device 176) the valve stem 132 enters the valve body 102.

The purpose of the stalk 190 of the base cap 172 is illustrated in FIG. 12. As shown in the figure, the stalk 190 extends vertically upwards from the centre of the base cap 172. The valve assembly 160 is shown on the end of the stalk 190. The stalk 190 may be integral with the valve body 102 of the valve assembly 160 (as shown in FIG. 12) or, alternatively, the valve assembly 160 may be mounted on a stalk 190.

To assemble the complete aerosol container 176, the base cap 172, with the stalk 190 and valve assembly 160 attached, is fitted to the tubular container 166. The valve stem 132 extends through the opening 168 provided in the neck 178 of the tubular container 166. A snap-fit is provided between the base cap 172 and the tubular container 166 as previously described. Once formed, the snap-fit joint between the base cap 172 and container 166 may be jacketed by over-molding 184. The fully assembled aerosol device 176, including the over-molding 184, is illustrated in FIG. 13. The over-molding 184 may be done by any one of the means including but not limiting to fusing, molding, welding, ultra sound welding, shrink sleeve and the like.

Once assembled, the base cap 172, through the inclusion of the stalk 190, provides support for the valve assembly 160 that may be provided as a separate part or as an integral part. The valve assembly 160 is pressed into position in the neck 178 of the container 166 by the stalk 190. This allows the seat gasket 156 to form a seal with the area around the opening 168 in the neck 178 of the container 166 to provide a hermetically sealed container 176. The valve assembly 160 is held in place without the need for crimpling of a valve ferrule, and the sealing is provided by the gasket 156 and the snap-fit between the container body 166 and base cap 172. If a metal is used as the material of construction an elastomer may be required to snap fit the base cap to the main body.

Thus the present invention provides, a pre-assembled, hermetically sealed device, wherein the only task that needs to be done is filling the device with the required amount of medicament. As a result it reduces the production time considerably.

When the valve stem 132 is pressed, the fluid inside the container 166 enters the valve chamber 116 through small apertures and it is expelled through the stem 132 in the form of a fine mist or spray. Although a particular valve assembly is shown and described, it should be apparent that the concept described would also work with alternative vale assemblies. The valve or valve assembly may be selected from the different types valves/valve assemblies including but not limiting to vertical valve assemblies, toggle action aerosol valve, the female aerosol valve, ferrule type aerosol valve, one-shot valve and the like.

A suitable nipple or other actuating devices may be mounted on the mouth of the annular space at the extremity of the valve stem 132 distal from the spring element 154 for directing the pressurized contents of the container as desired by the user.

The invention consists of a number of distinct features which are, for the sake of brevity, described in the context of two preferred embodiments. It should be appreciated that the various features are advantageous in their own right and may be provided separately or in a suitable alternative combination.

The container body 66,166 of the present invention must be capable of withstanding the vapour pressure of the propellant used. The container 66,166 is preferably transparent, and is formed from a polysulphone, since this has been found to be appropriate for forming a body capable of withstanding the vapour pressure of a propellant used. Alternative materials may, however, also be considered. Materials which may be used include, but are not limited to, metals (aluminum, stainless steel), polymers/plastics (polycarbonate; polyethylene terephthalate (PET); polyethersulphone, polysulphone, thermoplastic polycarbonates; copolymers of ethylene and C1 C6 mono- or di-unsaturated monomers; ethylene based polymers including ethylene/vinyl acetate, ethylene acrylate, ethylene methacrylate, ethylene methyl acrylate, ethylene methyl methacrylate, ethylene vinyl acetate carbon monoxide, and ethylene. N-butyl acrylate carbon monoxide; polybutene-1; high and low density polyethylene; polyethylene blends and chemically modified polyethylene; polybutadiene rubber; polyamides; polyesters such as polyethylene terephthalate, polyethylene naphthalate, polybutylene terephthalate; atactic polyalphaolefins including atactic polypropylene, polyvinylmethylether and others; thermoplastic polyacrylamides; polyacrylonitrile; copolymers of acrylonitrile and other monomers such as butadiene styrene; polymethyl pentene; polyphenylene sulphide; aromatic polyurethanes; styrene-acrylonitrile; acrylonitrile-butadiene-styrene); and glass.

A number of the materials listed above are also suitable for manufacturing other parts of the aerosol device 76. The material for making the valve may be at least one selected from a group of materials consisting of metals; glass; thermoplastics materials including but not limiting to polymers such as PET, polycarbonates and the like and any combinations thereof. Alternatively, the valve assembly may be made of acetyl or polyester, Hytrel®, or the like.

The container 66 made from the aforementioned materials is preferably entirely transparent, but may be partially or fully opaque to block the passage of light therethrough. The container 66 may further be provided with markings (etchings) indicative of the number of doses of a formulation remaining in the container. This is especially useful where the container 66 is formed of a transparent or translucent material, since the contents of the container are visible; and the markings would provide a clear indication of the quantity of substance remaining after periodic use.

The elastomeric materials used in the aerosol device may be at least one of nitrile, butyl, chloroprene, EPDM, TPE, HNBR, POE, chlorobutyl, and bromobutyl or any other thermoplastic elastomer.

The aerosol device according to the present invention makes minimum use of elastomers and is highly leak resistant. Accordingly, a wide variety of propellants may be used. These include, but are not limited to, CFC Propellants (e.g. dichlorodifluoromethane, monofluorotrichloromethane, dichlorotetrafluoroethane and combinations thereof), propane, butane, isobutene, isopropane, LPG, HFA Propellants (e.g. Tetrafluoroethane (HFA134a), Heptafluoropropane (HFA227) and combinations thereof) with or without any polar or non polar co solvents and/or surface active agents.

The inner surface of the container may optionally be anodized, lacquer-coated and/or plastic-coated with suitable coating materials, so as to render it anti-adherent. Various materials are suitable for coating the inner surface of a container. These may be organic (polymeric coats such as polyamides) or inorganic in nature like epoxyphenolic, PFA, FEP/PES, Teflon, Silicon, ethylene, and xylan.

The various component parts of the aerosol device may be made by any appropriate manufacturing techniques. In the case of parts made of a plastics material, molding techniques including, but not limited to, injection molding, two stage blow molding, compression molding, transfer molding, extrusion molding, blow molding, rotational molding or thermoforming are preferred.

The container according to the present invention may be of any suitable shape, but is preferably of the shape as shown in the drawings so as to ensure that almost all of a substance contained therein is delivered during operation.

1-75. (canceled)

76. An aerosol device comprising a container with an opening at one end, a valve assembly supported within the container, and sealing means for providing a seal between the valve and the container.

77. The aerosol device according to claim 76, further comprising means for retaining the valve assembly in the container, wherein said means for retaining the valve assembly in the container is separate from said sealing means.

78. The aerosol device according to claim 77, wherein the means for retaining the valve assembly in the container comprises a cap member, the cap member being secured to the container in order to retain the valve assembly in position and being the cap member is secured to the outside of the container.

79. The aerosol device according to claim 78, wherein the valve assembly is received in the opening in said one end of the container.

80. The aerosol device according to claim 79, wherein the valve assembly is provided with a sealing element around its entire periphery. the valve assembly closing the opening in said one end of the container.

81. The aerosol device according to claim 80, wherein a circumferential ledge is provided on the inside of the container, adjacent the opening, to support the valve assembly in the container.

82. The aerosol device according to claim 81, wherein the cap member is provided with an aperture and a valve stem of the valve assembly extends through the aperture in the cap member when the aerosol device is assembled.

83. The aerosol device according to claim 78, wherein the cap member closes the opening in said one end of the container.

84. The aerosol device according to claim 83, wherein the valve assembly is supported on a stalk extending from of the cap member, the stalk extending through the interior of the container when the aerosol device is assembled, to support the valve assembly in position at the end of the container opposite the opening.

85. The aerosol device according to claim 84, wherein an aperture is provided in the end of the container opposite the opening, and a valve stem of the valve assembly extends through the aperture in the container when the aerosol device is assembled.

86. The aerosol device according to claim 78, wherein the cap member is crimped to the container.

87. The aerosol device according to claim 78, wherein the container is formed, at least in part, from transparent or translucent material.

88. The aerosol device according to claim 87, wherein the container is provided with markings to indicate the amount of a substance in the container.

89. The aerosol device according to claim 87, wherein the container comprises a polymer of a sulphone (a polysulphone).

90. The aerosol device according to claim 78, wherein, in use, a flat base portion is provided on the interior of the container, the flat base portion being provided by the valve assembly.

91. An aerosol device comprising a container formed at least in part from transparent or translucent material.

92. The aerosol device according to claim 91, wherein markings are provided on the container to indicate the amount of a substance in the container.

93. The aerosol device according to claim 91, wherein, in use, a flat base is provided in the interior of the container, the flat base being formed by a valve assembly.

94. The aerosol device according to claim 93, wherein apertures are provided adjacent the flat base to permit a substance held in the container to enter a valve assembly.

95. The aerosol device according to claim 91, wherein the container comprises a polymer of a sulphone (a polysulphone).

96. The aerosol device according to claim 95, wherein the polysulphone comprises a number, x, of repeating sulphone monomer units.

97. The aerosol device according to claim 96, wherein the sulphone monomer comprises the moiety

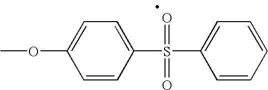

98. The aerosol device according to claim 97, wherein the polysulphone has the chemical formula

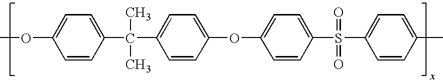

99. The aerosol device according to claim 98, wherein the polysulphone has a molecular weight ranging from around 30500 to around 35000.

100. The aerosol device according to claim 96, wherein x is an integer ranging from around 14000 to around 16000.

101. The aerosol device according to claim 78, wherein the entire container is transparent or translucent.

102. A Metered Dose Inhaler comprising an aerosol device according to claim 78.

103. A valve assembly for an aerosol device, the valve assembly being receivable in an opening provided in a container of the aerosol device to close said opening, wherein means are provided on the valve assembly to form a seal between the valve assembly and a container.

104. The valve assembly according to claim 103, wherein the means to form a seal is provided around the entire periphery of the valve assembly.

105. The valve assembly according to claim 104, comprising a flat portion which, in use, faces towards the interior of a container, the flat portion being formed by a body part of the valve assembly.

106. The valve assembly according to claim 105, wherein the valve assembly comprises channels immediately adjacent the flat portion providing access to the interior of the valve assembly.

107. The valve assembly according to claim 106, wherein the component parts of the valve assembly are arranged in a valve body and contained therein by a sealing piece to provide a self contained valve assembly.

108. The valve assembly according to claim 107, wherein the component parts of the valve assembly comprise a valve stem, a spring and a valve chamber; wherein a space is provided between the valve stem and valve chamber, said space defining one dose to be released when the valve is actuated.

109. The valve assembly according to claim 108, wherein the component parts of the valve assembly further comprise a seat gasket positioned between the valve chamber and the sealing piece, wherein the seat gasket surrounds the valve stem and is capable of sealing an aperture provided in the valve stem.

110. An aerosol device comprising a valve assembly according to claim 103.
